(19) United States
(12) Patent Application Publication
(10) Pub. No.: US 2026/0296986 A1
(43) Pub. Date: Oct. 1, 2026

(54) INCREASING CARBON STORAGE CAPACITY AND POLLUTANT REMEDIATION IN SOILS USING APPLIED HYDROGEL METHODS

(71) Applicant: University of Washington, Seattle, WA (US)

(72) Inventors: Mari-Karoliina Henriikka Winkler, Seattle, WA (US); Korena Mafune, Seattle, WA (US); Pieter Michiel Rogier Candry, Seattle, WA (US)

(73) Assignee: University of Washington, Seattle, WA (US)

(21) Appl. No.: 19/477,989

(22) PCT Filed: Apr. 25, 2024
(86) PCT No.: PCT/US2024/026322

§ 371 Date: Oct. 23, 2025

Related U.S. Application Data
(60) Provisional application No. 63/498,427, filed on Apr. 26, 2023.

(51) Int. Cl. C05F 11/08; A01G 24/10; A01G 24/22; B09C 1/08; B09C 1/10; C05D 9/00; C05G 3/80; C05G 5/18

(52) U.S. Cl. CPC C05F 11/08; A01G 24/10; A01G 24/22; B09C 1/08; B09C 1/105; C05D 9/00; C05G 3/80; C05G 5/18

(57) ABSTRACT

A hydrogel comprising a non-biological C-capturing substance and a microbial consortium, and methods for using the hydrogel for carbon sequestration in a soil, for enhancing plant growth, plant productivity, and/or crop yield, and for enhancing remediation of a pollutant from a soil.

### CROSS-REFERENCE TO RELATED APPLICATION

This application claims the benefit of U.S. Patent Application No. 63/498427, filed Apr. 26, 2023, expressly incorporated herein by reference in its entirety.

### BACKGROUND

Currently, conventional fertilizer prices are soaring, and their use remains unsustainable (e.g., water pollution and GHG emissions) leading to $157 billion of damage per year in the US alone. In 2020, ammonia costs were about $400/ton with a steep increase to about $1,500 in 2021, and costs are forecasted to exponentially increase in upcoming years. Non-biological C-sorbing substances and biofertilizer can offset some of these costs. In addition, soil systems have the potential to gain profit through C credits, as agricultural land in the US holds the capacity to sequester about 650 million metric tons of CO₂ every year, which could offset up to 11% of US GHG emissions annually. Besides agricultural land, other terrestrial surfaces such as forests and wetlands could offer additional C storage capacities. Therefore, if the aggregate of natural and agricultural soils were managed using climate-smart agricultural practices, they could act as powerful net C sinks, with the potential to store over a billion tons of C each year. At the same time, N₂O emissions (about 300× the warming potential of CO₂) are barely controlled. Over 60% of global anthropogenic N₂O stems from fertilized agricultural soils and fertilized soils change the microbial community, hence increasing N₂O emissions. In addition, other terrestrial systems like natural wetlands account for 4-6% of global land area, release 20% of global CH₄, and store approximately 30% of global soil organic carbon.

Globally, soils contain about 2,400 Gt of C to a 2 m depth, which exceeds the total mass of C in vegetation and the atmosphere combined. Agricultural landscapes represent 38% of the earth's terrestrial surface and are typically depleted in soil C compared to the native soils from which they were derived. This C depletion is due to reduced net primary productivity, biomass export, nutrient depletion, anthropogenic disturbances, and erosion.

A need exists for materials and methods for controlling C and N cycling for climate-smart crop production, restoring the soil with C lost over decades due to unsustainable agricultural practices, and increasing crop yield. The present disclosure seeks to fulfill these needs and provide further related advantages.

### SUMMARY

In one aspect, the present disclosure provides a hydrogel, comprising a non-biological C-capturing substance and a microbial consortium. In certain embodiments, the non-biological C-capturing substance is a biochar pyrolyzed from woody biomass, a biochar pyrolyzed from vegetative biomass, a biochar pyrolyzed from herbaceous biomass, a biochar pyrolyzed from agricultural waste products, a biochar pyrolyzed from sewage sludge, a biochar pyrolyzed from food waste, or a biochar pyrolyzed from animal manure, a zeolite, or a clay. In certain embodiments, the microbial consortium is a bacterial consortium, an archaeal consortium, a fungal consortium, an algal consortium, or a combination thereof.

In another aspect, the present disclosure provides a method for carbon sequestration (e.g., capture, stabilization, and storage) in a soil using a hydrogel as described herein.

In a further aspect, the present disclosure provides a method for enhancing plant growth, plant productivity, and/or crop yield using a hydrogel as described herein.

In another aspect, the present disclosure provides a method for enhancing remediation of a pollutant from a soil using a hydrogel as described herein.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 compares soil organic matter (i.e., soil organic carbon) in soils with sunflower as host plants that had microbial-biochar hydrogels applied.

FIG. 2 compares para-cresol remediation in soils with sorghum as host plant. The blank control (blk) was just treated soil with no hydrogels, the negative control (Neg_Cnt) was plant and soil with no hydrogels, positive control 1 (P_Cnt1) was just hydrogel-biochar with no organisms, positive control 2 (P_Cnt2) was loose inoculum with no char or hydrogel, and Treatments 1 through 4 (Trt1-Trt4) are various combinations of hydrogel-char-microbe.

FIG. 3 compares zinc leachate from soils with sunflower as host plant that had microbial-biochar hydrogels applied.

FIG. 4 compares copper leachate from soils with sunflower as host plant that had microbial-biochar hydrogels applied.

FIG. 5 compares soil lead in soils with sunflower as host plant that had microbial-biochar hydrogels applied.

FIG. 6 compares soil aluminum in soils with sunflower as host plant that had microbial-biochar hydrogels applied.

FIG. 7 compares sunflower above-ground biomass in soils that had microbial-biochar hydrogels applied.

FIGS. 8A-8C are microscopic images of bacterial-fungal-algal-char hydrogels. FIGS. 8A and 8C show biochar and algae near the edge of the hydrogel construct. FIG. 8B shows a more central location within the bead, the asterisk (*) shows arbuscular mycorrhizal spores in the hydrogel construct surrounded by algal and bacterial cells.

### DETAILED DESCRIPTION

The present disclosure provides an eco-friendly hydrogel that encases biochars with mixed bacterial-archaeal-fungal consortia that can be applied to forests, grasslands, shrublands, croplands, and wetlands at planting or anytime during the plant life cycle to enhance plant growth, health, and/or yield while simultaneously providing the benefit of carbon stabilization and long-term storage and/or remediation of soil pollutants. The hydrogel provides a format for rapid and gentle entrapment for aerobic and anaerobic archaea, bacteria, fungi, exudates, as well as solid material such as a biochar; thereby providing a matrix in which prokaryotic cells and fungal structures (e.g., spores and hyphae) can be protected, and substrates are readily and advantageously available within close vicinity of the consortia and plant host. The co-entrapment of biochar substrates with mixed prokaryotic-fungal consortia in hydrogels promotes the establishment of ecological micro-niches on the biochar surface structure and within pores that can increase carbon and/or pollutant (e.g., heavy metals and organic contaminants) sorption to the biochar, promotes nutrient exchange from the biochar to the consortia to the plant host, helps counteract any negative impacts biochar can have on plant nutrient uptake, and minimizes biochar displacement by wind and water erosion.

The present disclosure provides a hydrogel that encases non-biological C-sorbing substances (e.g., biochar pyrolyzed from various types of biomass including woody biomass, vegetative biomass, herbaceous biomass, agricultural waste biomass, sewage sludge biomass, and animal manure biomass) in eco-friendly hydrogels with beneficial fungal-bacterial-archaeal consortia to increase soil carbon (C) sequestration and stabilization leading to long-term C storage. These improvements can include increased plant biomass and crop yield in agricultural ecosystems, increased soil health and host plant resilience in forested, wetland, shrubland and grassland ecosystems with established plant communities, and increased seedling emergence and overall plant health in ecosystems being restored. The hydrogels described herein also hold the power to concurrently decrease microbially produced CO₂ and N₂O emissions, provide bioprotection (e.g., biocontrol) for native plants or crops, and remediate soil pollutants, or be designed to specifically target one or any combination of these functions.

In practice, the hydrogels described herein increase retention time of particles and microbes in soils, improve soil moisture retention (i.e., reducing drought stress), avoid displacement of biochar, and provide an environment where anaerobic and aerobic microorganisms can be protected from environmental stressors while retaining functional integrity in close vicinity of biochar particles. The hydrogel described herein with encased microbial communities are applied when seeds arc planted or at any time during the plant's lifecycle, which is dependent on the given ecosystem. The community encased in the hydrogels includes organisms adaptive to different climatic conditions. For example, the hydrogels encase bacteria and fungi that can be metabolically active in warmer months during periods of increased soil aeration and plant photosynthesis, or in colder months when soil aeration decreases, and anaerobic pockets can develop. Bacteria, fungi, and archaea that increase the functional versatility of the community year-round include (a) N-fixing bacteria that can excrete atmospherically fixed N₂ in a plant available form and have plant growth promoting (PGP) effects, (b) PGP fungal endophytes (including dark septate endophytes) and mycorrhiza that synergistically interact with prokaryotes to allocate nutrients to the plant host, (c) phosphate and potassium solubilizing bacteria that increase the amount of plant available nutrients in soil and have plant growth promoting effects, (d) mycorrhizal helper bacteria that promote mycorrhizal establishment and function, (e) phytohormone producing bacteria that have PGP effects, (f) bacteria and fungi that can act as bioprotectants to prevent pests and pathogens, and (g) ammonium oxidizing archaea (AOA) that can obtain excess ammonium and prevent ammonium oxidizing bacteria from thriving, hence reducing net N₂O emissions.

The hydrogels described herein include various biochar-hydrogel combinations that can facilitate C stabilization that results in long-term C storage and pairs it with an eco-friendly biofertilizer that can (a) alleviate usage of synthetic fertilizer in croplands, (b) increase seedling establishment and plant vigor in a range of ecosystems (forests, croplands, grasslands, wetlands), (c) replenish soil health in ecosystems with established plant communities, and (d) incorporate other beneficial microbes. Additionally, non-biological C-sorbing substances can lock up pollutants in soils, and therefore introduce the additional benefit of soil remediation. The application of the biochar-hydrogel technology described herein promotes plant productivity, adds in or retains a native soil community that promotes soil health, produces less N₂O, and potentially less methane, and enhances C sequestration and stabilization at the biochar-hydrogel-soil interface.

In one aspect, the present disclosure provides a hydrogel, comprising a non-biological C-capturing substance and a microbial consortium. The hydrogels described herein provide for adding bioremediating capacities, enhancing plant health and/or growth, and sequestering and stabilizing carbon in soils for long-term storage.

The hydrogels described herein are a cross-linked 3D network of polymers that can absorb and release water to increase moisture retention and decrease the loss of nutrients through leaching. The hydrogels are fabricated to be environmentally friendly by using biodegradable polysaccharides, such as chitosan, starch, and sodium alginate. In certain embodiments, the hydrogels encapsulate diazotrophic bacteria, plant growth promoting bacteria and archaea, ammonia oxidizing archaea, fungal endophytes, and mycorrhiza to increase C sequestration, nutrient uptake, and plant productivity (including but not limited to plant growth, crop yield, plant health, plant vigor), hence promoting sustainable and climate-friendly practices that can be applied to croplands, grasslands, shrublands, forests, wetlands, and ecosystems in the process of restoration. In certain embodiments, the hydrogels include biochar particles in the hydrogel to promote and increase carbon capture and soil remediation efforts, but also to reduce their displacement by wind, erosion, and water movement to avoid any unintended consequences, such as microbial/particle dispersal and risk of human inhalation. In certain embodiments, highly porous biochar's are used because they offer a niche for fungi, bacteria, and archaea to thrive. The hydrogel-biochar environment provides aerobic and anaerobic environments that allow both aerobic and anaerobic bacteria to be involved in carbon capture mechanisms. The hydrogel approach described herein allows for mixing and matching communities and biochar types and keep them close to each other and the plant rhizosphere, while also reducing biochar particle displacement by wind erosion and water movement.

Suitable non-biological C-capturing substances include a biochar pyrolyzed from woody biomass, a biochar pyrolyzed from vegetative biomass, a biochar pyrolyzed from herbaccous biomass, a biochar pyrolyzed from agricultural waste products, a biochar pyrolyzed from sewage sludge, a biochar pyrolyzed from food waste, or a biochar pyrolyzed from animal manure, a zeolite (e.g., aluminosilicates of the alkaline and alkaline-earth metals such as, but not limited to, analcime, chabazite, clinoptilolite, erionite, ferrierite, heulandite, laumontite, mordenite, and phillipsite), or a clay (e.g., kaolinite, smectite, chlorite, micas, baileychlore, brindleyite, corrensite, sudoite, and tosudite).

In certain embodiments, the biochar is a biochar pyrolyzed from woody biomass, vegetative biomass, or herbaceous biomass.

The hydrogel described herein includes a microbial consortium. Suitable microbial consortia include a bacterial consortium, an archaeal consortium, a fungal consortium, an algal consortium, or a combination thereof. In certain embodiments, the microbial consortium is a mix of two or more of the consortia, such as a bacterial-algal consortium or a bacterial-fungal-algal consortium.

It will be appreciated that the algal consortium is a source of carbon and nitrogen to provide the community with N/C rich components, such as (but not limited to) guanidine, amino acids and/or urea or a combination thereof to slow growth of nitrifying strains, such as complete ammonium oxidizing bacteria (comammox) and ammonium oxidizing bacteria (AOB), by which plants will have more time to access nitrogen hence also avoiding nitrogen leaching into the ground water.

The hydrogel encases different types of biochars in eco-friendly hydrogels with beneficial fungal-bacterial-archaeal consortia to take advantage of the underlying mechanisms that lead to an increase in soil C sequestration and stabilization, a decrease in microbial produced CO₂ and N₂O emissions, an improvement in plant health and productivity, and removal of soil contaminants. To maximize the functionality of beads, a year-round community made up of a “warm-season community” promoting plant growth and “cold-season community” enabling carbon breakdown and sequestration. The latter would remain dormant or less active during warmer months when the soil is well aerated and becomes more functional in the winter (or late fall/early spring). During winter, anacrobic pockets develop within the hydrogel/charcoal to enable the winter community's anaerobic capacities for carbon storage and sequestration. This way an efficient biofertilizer is provided that promotes high crop yield with minimal emission footprint as well as a sustainable capture of carbon in seasons where crop growth is limited but soil activity will continue.

Warm-season strains include: (a) N-fixing bacteria that can excrete atmospherically fixed N₂ in a plant available form (ammonium) and have plant growth promoting effects (b) mycorrhiza that provide bacteria with plant derived carbon and that take up nitrogen to provide it to the crop and (c) ammonium oxidizing archaea (AOA) that can pick up any remaining ammonium to limit ammonium oxidizing bacteria growth and reduce net N₂O emissions. The present disclosure provides a natural biofertilizer that maintains a healthy, functional soil community known to produce less N₂O and promote carbon storage in fungal biomass and necromass hence contributing to long-term storage of soil organic matter (SOM). Additionally, non-biological C-sorbing substances capture and store pollutants contributing to removal of soil pollutants. Any C leaking into the environment is retained by the biochar.

Cold-season strains include: (a) spores of a facultative anaerobic decomposing fungal strain capable of degrading the bacterial and archaeal biomass established in the summer to either store it in their cell biomass hence promoting necromass formation or passing it on as small organic acids (e.g., acetic and lactic acids) to (b) chain elongating bacteria (CEB) encapsulated as spores. These CEB convert the released organic acids to medium chain carboxylic acids (MCCA, 6-12 carbon organic acids), which remain in the soil on longer term due to their toxicity to microbes at elevated concentrations as well as interactions with soil minerals. Chain elongating bacteria can outcompete methanogens which will reduce methane release from soil systems. Facultative anaerobic saprotrophic filamentous fungi translocate C in deeper/more reduced pockets within the charcoal where fungal metabolites can drive cross-feeding interactions to chain elongators that contribute to C sequestration via MCCA production. The contribution of these MCCA to the formation of refractory C then provides an avenue for long-term C sequestration. Some strains may function as both summer and winter strains such as the chain elongator Clostridium kluyveri which is capable of both MCCA production and N fixation.

In certain embodiments, the microbial consortia comprise N-fixing bacteria and/or bacteria, fungi, and/or archaea that have plant growth promoting effects including phytohormone production, nutrient solubilization, and increased resistance to pathogens.

In certain embodiments, the microbial consortia comprise bacteria that can solubilize or source macro and micronutrients from the soil.

Microbial consortia useful in the hydrogel include bacterial consortia. Representative bacterial consortia include:
N-fixing bacteria that excrete atmospherically fixed N₂ in a plant available form (ammonium) and bacteria, fungi, and/or archaea that have plant growth promoting effects including phytohormone production, nutrient solubilization, and increased resistance to pathogens;
bacteria that can solubilize or source macro (e.g., K⁺, P, Na⁺, Mg²⁺) and micro (e.g., Cu²⁺, Mn²⁺, Zn²⁺) nutrients from soil;
mycorrhizal helper bacteria that aid in mycorrhizal pre-symbiotic growth, stimulate mycorrhizal establishment, increase plant receptivity to mycorrhizal establishment, and/or promote mycorrhizal survivability and reproduction; and
chain elongating bacteria (CEB), optionally encapsulated as spores, that generate long chain carboxylic acids.

Microbial consortia useful in the hydrogel may also include fungal consortia. Representative fungal consortia include:
arbuscular mycorrhizal fungi (AMF) that allocate resources (e.g., macronutrients, micronutrients, and water) to the plant in exchange for plant derived carbon (e.g., photosynthates) that can be translocated and provided to the other consortia community members or get captured by non-biological C-sorbing substances or get stored in biomass and necromass;
ectomycorrhizal fungi (EMF) that allocate resources (e.g., macronutrients, micronutrients, and water) to the plant in exchange for plant derived carbon (e.g., photosynthates) that can be translocated and provided to the other consortia community members or get captured by non-biological C-sorbing substances or get stored in biomass and necromass;
ericoidal mycorrhizal fungi (ErM) that allocate resources (e.g., macronutrients, micronutrients, and water) to the plant in exchange for plant derived carbon (e.g., photosynthates) that can be translocated and provided to the other consortia community members or get captured by non-biological C-sorbing substances or get stored in biomass and necromass; and
melanized fungi (e.g., dark septate endophytes) that increase plant resistance to drought, increase the plant resistance to and capability to degrade pollutants (e.g., heavy metals), and/or that allocate resources (e.g., macronutrients, micronutrients, and water) to the plant in exchange for plant derived carbon (e.g., photosynthates) that can be translocated and provided to the other consortia community members or captured by non-biological C-sorbing substances or get stored in biomass and necromass.

In certain embodiments, the microbial consortia include ammonium oxidizing archaea (AOA) that produce less N₂O than typical ammonium oxidizers (AOB) present in ammonium laden soils such as agricultural.

In certain embodiments, the microbial consortia include aerobic methanotrophs and/or nitrate dependent anaerobic methane oxidizers capable of removing methane.

In certain embodiments, the microbial consortia comprise arbuscular mycorrhizal fungi (AMF).

In certain embodiments, the microbial consortia comprise ectomycorrhizal fungi (EMF).

In certain embodiments, the microbial consortia comprise ericoidal mycorrhizal fungi (ErM).

In certain embodiments, the microbial consortia comprise mycorrhizal helper bacteria.

In certain embodiments, the microbial consortia comprise of melanized fungi.

In certain embodiments, the microbial consortia comprise spores, conidia, microsclerotia, and/or hyphal fragments of an aerobic, anaerobic, microaerobic, and facultative decomposing fungal strain as a spore or as any active form during its life cycle capable of degrading bacterial and/or archaeal biomass.

In certain embodiments, the microbial consortia comprise chain elongating bacteria (CEB), optionally encapsulated as spores.

The hydrogels described herein may advantageously include a pollutant-remediating substance. Useful pollutant-remediating substances include non-biological substances or biological substances. Representative non-biological pollutant-remediating substances include zeolites, biochar, clays, silicates, oxide minerals, vermiculites, and apatite. Representative biological pollutant-remediating substances include certain species of the microbial consortium. Non-biological C-capturing substances can increase C storage and can also bind and remediate pollutants.

The hydrogels described herein may advantageously include an additional component. Suitable additional components that are advantageously be included in the hydrogel include mineral nutrients (micronutrients and macronutrients), natural fertilizers (e.g., compost, biochar can also act as a fertilizer amendment in certain cases), chemical substances, nitrification inhibitors, vitamins, enzymes, carbohydrates, proteins, hormones, and biocontrol pesticides.

Other suitable additional components that are advantageously be included in the hydrogel include sources of nitrogen (N), phosphorus (P), calcium (Ca), potassium (K), sulphur (S), magnesium (Mg), boron (B), zinc (Zn), manganese (Mn), iron (Fe), copper (Cu), molybdenum (Mo), and chlorine (Cl).

Further suitable additional components that are advantageously be included in the hydrogel include lignin, cellulose, hemicellulose, B-complex vitamins, vitamin D, vitamin C, auxins, cytokinins, nitrapyrin, dicyandiamide, 3,4-dimethylepyrazole phosphate, biosolids, biochar, compost, sawdust.

In certain embodiments, the hydrogels described herein have a size from about 1 to about 7 millimeters in diameter.

In certain embodiments, the hydrogels described herein are derived from a biocompatible (biodegradable) crosslinked polymer. Biodegradable hydrogel bead can be fabricated using biodegradable compositions including, but not limited to, cellulose, carrageenan, hyaluronic acid, polysaccharides (e.g., alginate, starch, agarose), chitosan, fibrin, and/or proteins (e.g., gelatin, collagen) or a combination thereof.

Useful crosslinkers include various chemical compounds including, but not limited to, calcium²⁺, iron²⁺, copper²⁺, barium²⁺, aluminum³⁺, iron³⁺, potassium¹⁺, or other suitable cations for ionotropic gels, or N,N′-methylenebisacrylamide, epoxy compounds, aldehyde compounds, ethylene glycol diacrylates, PEG diacrylates, PEG dithiols, tannic acid boric acid and substituted boronic acids, for covalently crosslinked gels. Biodegradable gel network-forming materials can comprise alginate, heparin, chitosan, carrageenan, dextran, gelatin, gellan gum, agar, fibrin, and starch. Non-biodegradable gel materials can comprise PEG acrylates, PEG diacrylates, multi-arm PEG acrylates, multi-arm PEG norbornenes, acrylamides, hydroxy-substituted acrylates, acidic acrylates (e.g., acrylic acids), amine-substituted acrylates (e.g., 2-(diethylamino)ethyl methacrylate), and polyvinyl alcohol.

In certain embodiments, the hydrogel described herein is a Na-alginate hydrogels crosslinked with calcium chloride. In other embodiments, the Na-alginate hydrogels crosslinked with calcium chloride further includes starch to provide starch-alginate hydrogels.

The preparation of representative hydrogels of the disclosure and their field application is described in Example 1.

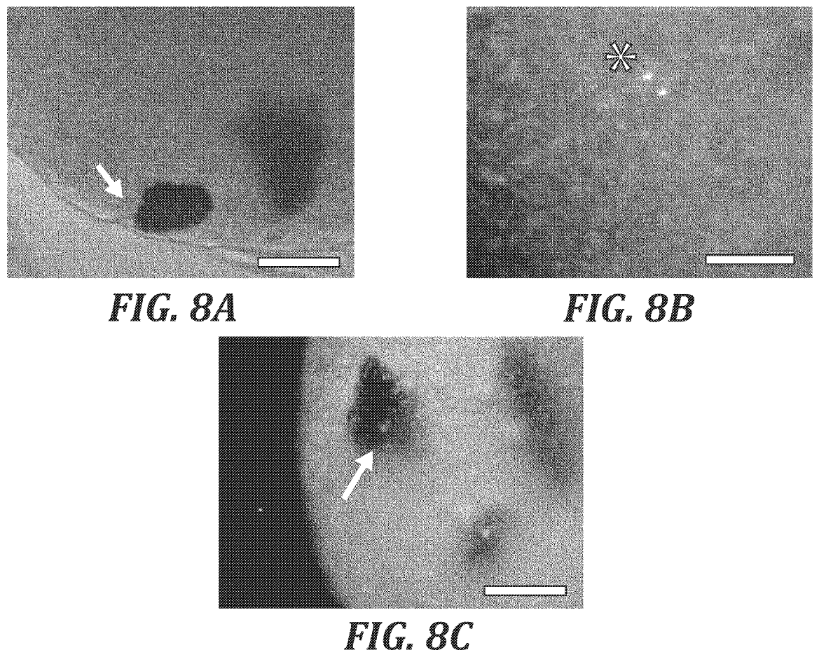

FIGS. 8A-8C are microscopic images of a representative hydrogel of the disclosure: a bacterial-fungal-algal-char hydrogel. FIGS. 8A and 8C show biochar and algae near the edge of the hydrogel construct. FIG. 8B shows a more central location within the bead, the asterisk (*) shows arbuscular mycorrhizal spores in the hydrogel construct surrounded by algal and bacterial cells. These images demonstrate that the hydrogel mixed consortium can include algal species (e.g., microalgae), as well as bacteria, fungi, archaea, and non-biological substances.

In other aspects, the present disclosure provides method for using the hydrogels described herein.

In one aspect, the present disclosure provides a method for carbon sequestration (e.g., capture, stabilization, and storage) in a soil. In the method, a hydrogel as described herein is applied a soil (e.g., sand, sediment, or peat) in either a managed system (e.g., agricultural land) or a natural system (e.g., wetland or forest). In the method, plants suitable for carbon sequestration include forest plants.

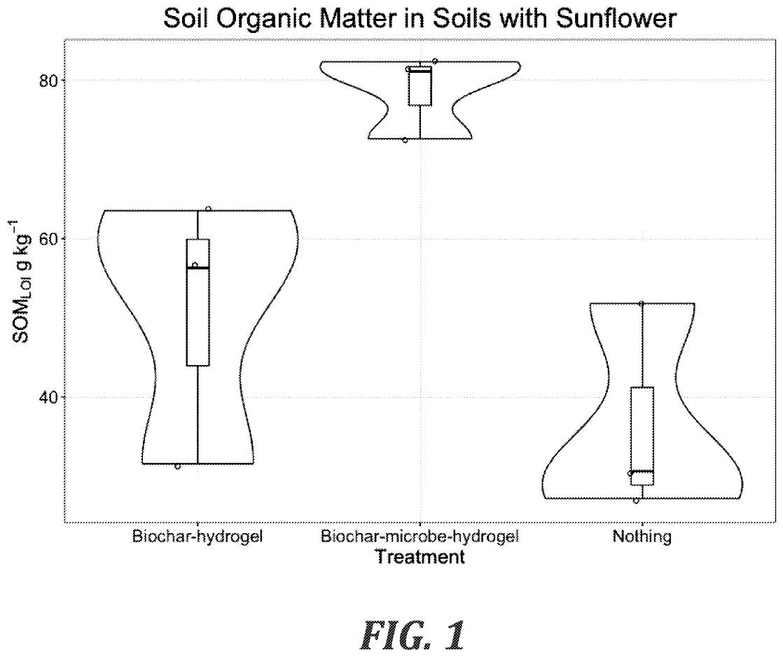

FIG. 1 compares soil organic matter (i.e., soil organic carbon) in soils with sunflower as host plants that had microbial-biochar hydrogels applied. Referring to FIG. 1, there was a significant increase (P<0.05) in soil organic matter in soils that had microbial-biochar hydrogels applied demonstrating that microbial-char embedded hydrogels can benefit soil carbon storage when applied to sunflower plants.

In another aspect, the present disclosure provides a method for enhancing plant growth, plant productivity, and/or crop yield. In the method, a hydrogel as described herein is applied to a soil at the time of planting or at a time during the plant lifecycle. In the above method, suitable plants that benefit from enhanced plant growth, plant productivity, and/or crop yield include bio-based crops (e.g., switchgrass and poplar), cash crops (e.g., tomato), grains (e.g., wheat), and fruit trees (e.g., cherry).

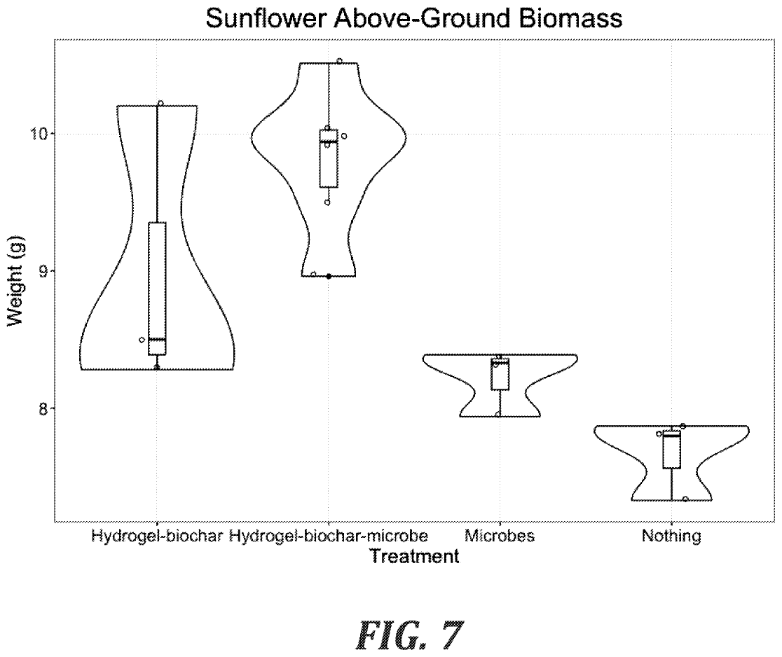

FIG. 7 compares sunflower above-ground biomass in soils that had microbial-biochar hydrogels applied. There was a significant increase (P<0.05) in above-ground biomass of sunflowers treated with hydrogel-microbial-biochar demonstrating that they improve plant growth compared to controls that did not receive the hydrogel-char biotechnology.

In a further embodiment, the present disclosure provides a method for enhancing remediation of a pollutant from a soil. In the method, a hydrogel as described herein is applied a soil in need of remediation.

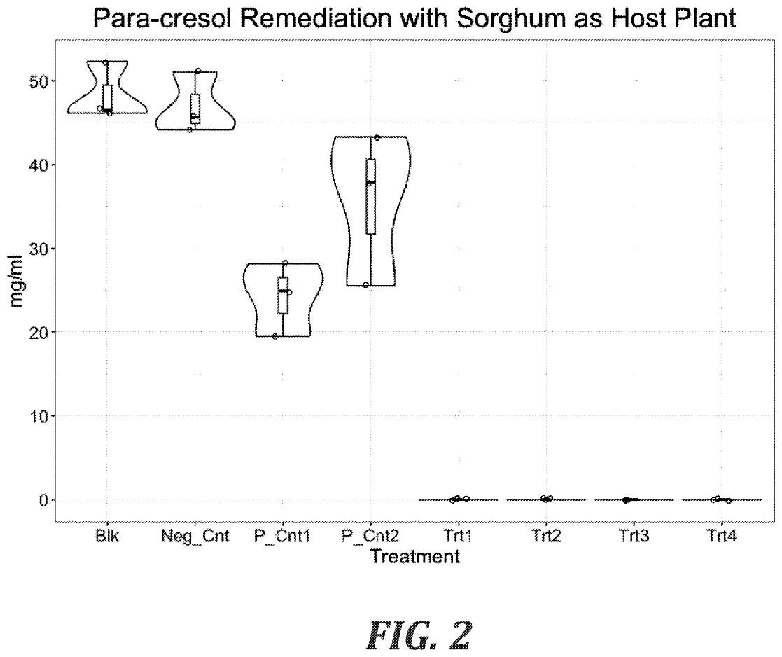

FIG. 2 compares para-cresol remediation in soils with sorghum as host plant. All of the hydrogel-microbial-biochar combinations successfully removed the phenol-derived toxin para-cresol from soils. Referring to FIG. 2, the blank control (blk) was just treated soil with no hydrogels, the negative control (Neg_Cnt) was plant and soil with no hydrogels, positive control 1 (P_Cnt1) was just hydrogel-biochar with no organisms, positive control 2 (P_Cnt2) was loose inoculum with no char or hydrogel, and Treatments 1 through 4 (Trt1-Trt4) are various combinations of hydrogel-char-microbe. This demonstrates that when biochars and microbes are encased together within the hydrogel construct, there is a significant effect (P<0.05) on p-cresol removal.

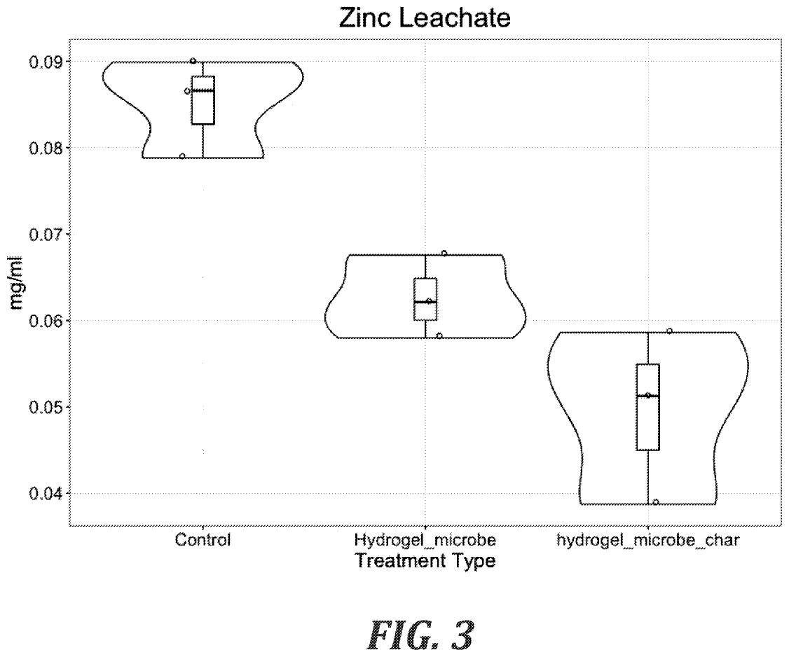

FIG. 3 compares zinc leachate from soils with sunflower as host plant that had microbial-biochar hydrogels applied. Referring to FIG. 3, there was a significant decrease (P<0.05) in zinc leaching from soils that had microbial-biochar hydrogels applied demonstrating that microbial-char embedded hydrogels can decrease the amount of this heavy metal leaching from soils that have sunflower planted.

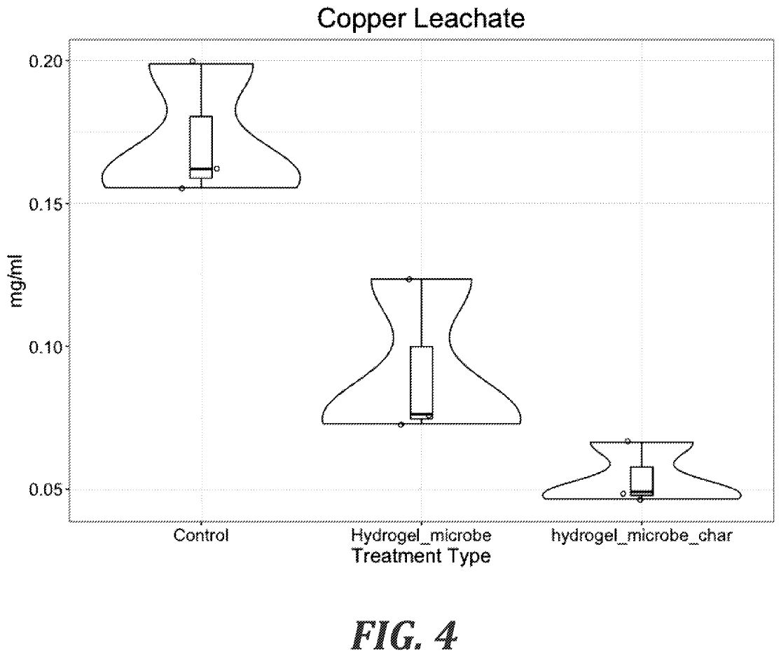

FIG. 4 compares copper leachate from soils with sunflower as host plant that had microbial-biochar hydrogels applied. Referring to FIG. 4, there was a significant decrease (P<0.05) in copper leaching from soils that had microbial-biochar hydrogels applied demonstrating that microbial-char embedded hydrogels can decrease the amount of this heavy metal leaching from soils that have sunflower planted.

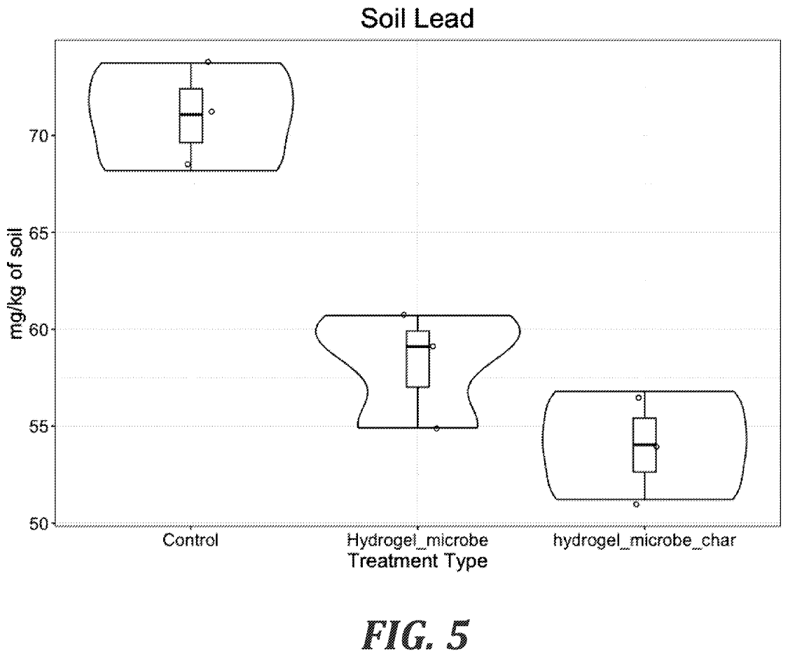

FIG. 5 compares soil lead in soils with sunflower as host plant that had microbial-biochar hydrogels applied. Referring to FIG. 5, there was a significant decrease (P<0.05) in the presence of lead extracted from soils that had microbial-biochar hydrogels applied demonstrating that microbial-char embedded hydrogels can decrease the amount of this heavy metal in soils that have sunflower planted.

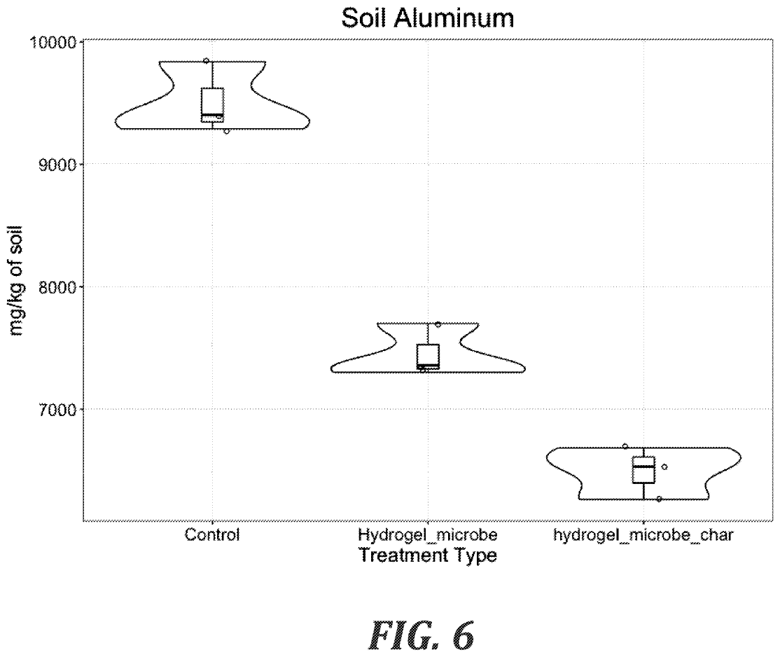

FIG. 6 compares soil aluminum in soils with sunflower as host plant that had microbial-biochar hydrogels applied. Referring to FIG. 6, there was a significant decrease (P<0.05) in the presence of aluminum extracted from soils that had microbial-biochar hydrogels applied demonstrating that microbial-char embedded hydrogels can decrease the amount of this heavy metal in soils that have sunflower planted.

The following examples are provided for the purpose of illustrating, not limiting, the disclosure.

### EXAMPLES

### Example 1

### The Preparation of and Field Application of Representative Hydrogels

In this example, the preparation of representative of hydrogels of the disclosure is described: (1) 2% sodium alginate hydrogel beads with a mixed microbial-char consortia and (2) boric acid-crosslinked PVA and calcium-crosslinked alginate hydrogels post-cured with sulfate.

To form the hydrogels described herein, a liquid polymer mixture is forced through Masterflex L/S tubing using a peristaltic pump, a pressurized syringe pump system, and/or an engineered system into a container that contains a cross-linking solution.

Cross-linking and polymer solutions can vary depending on the type of hydrogel being fabricated, but in general the two solutions are always prepared separately, and then the polymer solution of choice is dispensed into the cross-linking solution using the set-up of preference.

Sodium Alginate Hydrogel Beads with a Mixed Microbial-Char Consortia

To prepare 2% sodium alginate hydrogel beads with a mixed microbial-char consortia, the selected consortium and non-biological substance (e.g., fungal-bacterial with char) is mixed with 2% (w/v) of sterilized sodium alginate solution, and then placed on a magnetic plate stirrer for continuous suspension of spores, cells, and particles. Subsequently, one end of sterile L/S tubing connected to the pump and inserted into the container with the solution, organisms, and particles. The opposite end of the tubing is connected to a dispensing tip (can be different gauge sizes) that dispenses a drop into filter sterilized 2% CaCl₂ solution (w/v) which causes the drop to cross-link into a solid bead containing the consortia and particles.

### Boric Acid-Crosslinked Polyvinyl Alcohol (PVA) and Calcium-Crosslinked Alginate Hydrogels

Boric acid-crosslinked PVA and calcium-crosslinked alginate hydrogels post-cured with sulfate were prepared as described above for the sodium alginate hydrogel beads: a solution of 100 g L⁻¹ PVA, 10 g L⁻¹ sodium alginate, and the selected consortium and non-biological substance was pumped to a bead-dropping device consisting of ports with 24G needles dripping polymer droplets into a crosslinking solution containing 50 g H₃BO₃ L⁻¹ and 26.5 g CaCl₂·2H₂O L⁻¹ stirred with an overhead stirrer. After cross-linking, the SA-PVA beads were washed thoroughly with distilled water (DI) water to remove the crosslinking solution and incubated overnight in a solution of 73 g Na₂SO₄ L⁻¹. The ratio of total polymer-to-crosslinking solution was fixed at 1:10 across conditions.

### Field Application

Once the hydrogel is fabricated with the consortia and C-capturing substance of choice, they are ready to be applied to pot soils, field soils, or other plant growth media. The amount of hydrogel applied to soils will depend on the specific needs of the application. For example, if the goal of the hydrogel application was to improve plant N uptake and promote soil C storage on a ¼ hectare plot, 300 grams of biochar, 500+ fungal spores, and 5.0×10⁵ CFUs of N-fixing bacteria would be added to 1 liter of 2% sodium alginate, which would make 30,000 hydrogel beads to spread across the field. The amount administered does not have to be fixed and can range depending on the application need. For example, if the purpose was to promote plant growth and stabilize C, but there was heavy contamination, a larger quantity of beads could be fabricated to cover more soil area.

While illustrative embodiments have been illustrated and described, it will be appreciated that various changes can be made therein without departing from the spirit and scope of the invention.

1. A hydrogel, comprising a non-biological C-capturing substance and a microbial consortium.

2. The hydrogel of claim 1, wherein the non-biological C-capturing substance is a biochar pyrolyzed from woody biomass, a biochar pyrolyzed from vegetative biomass, a biochar pyrolyzed from herbaceous biomass, a biochar pyrolyzed from agricultural waste products, a biochar pyrolyzed from sewage sludge, a biochar pyrolyzed from food waste, or a biochar pyrolyzed from animal manure, a zeolite, or a clay.

3. The hydrogel of claim 1, wherein the microbial consortium is a bacterial consortium, an archaeal consortium, a fungal consortium, an algal consortium, or a combination thereof.

4. The hydrogel of claim 1, wherein the microbial consortia comprise N-fixing bacteria and/or bacteria, fungi, and/or archaea that have plant growth promoting effects.

5. The hydrogel claim 1, wherein the microbial consortia comprise bacteria that can solubilize or source macro and micronutrients from the soil.

6. The hydrogel of claim 1, wherein the microbial consortia comprise arbuscular mycorrhizal fungi (AMF).

7. The hydrogel of claim 1, wherein the microbial consortia comprise ectomycorrhizal fungi (EMF).

8. The hydrogel of claim 1, wherein the microbial consortia comprise ericoidal mycorrhizal fungi (ErM).

9. The hydrogel of claim 1, wherein the microbial consortia comprise mycorrhizal helper bacteria.

10. The hydrogel of claim 1, wherein the microbial consortia comprise of melanized fungi.

11. The hydrogel of claim 1, wherein the microbial consortia comprise ammonium oxidizing archaea (AOA), ammonium oxidizing bacterium (AOB) and/or complete ammonium oxidizing bacteria (comammox).

12. The hydrogel of claim 1, wherein the microbial consortia comprise spores, conidia, microsclerotia, and/or hyphal fragments of an aerobic, anaerobic, microaerobic, and facultative decomposing fungal strain as a spore or as any active form during its life cycle capable of degrading bacterial and/or archaeal biomass.

13. The hydrogel of claim 1, wherein the microbial consortia comprise chain elongating bacteria (CEB), optionally encapsulated as spores.

14. The hydrogel of claim 1, wherein the microbial consortia comprise aerobic and anaerobic methane oxidizing bacteria and or archaea.

15. The hydrogel of claim 1 further comprising a pollutant-remediating substance.

16. (canceled)

17. The hydrogel of claim 1 having a size from about 1 to about 7 millimeters in diameter.

18. The hydrogel of claim 1, wherein the hydrogel is derived from a biocompatible crosslinked polymer.

19. A method for carbon sequestration in a soil, comprising applying the hydrogel of claim 1 to a soil in either a managed system or natural system.

20. A method for enhancing plant growth, plant productivity, and/or crop yield, comprising applying the hydrogel of claim 1 to a soil at the time of planting or at a time during throughout the plant lifecycle.

21. A method for enhancing remediation of a pollutant from a soil, comprising applying the hydrogel of claim 1 to a soil.
